(19) United States
(12) Patent Application Publication
(10) Pub. No.: US 2025/0241799 A1
(43) Pub. Date: Jul. 31, 2025

(54) ENDOLUMINAL TREATMENT DEVICES AND RELATED METHODS

(71) Applicant: Boston Scientific Scimed, Inc., Maple Grove, MN (US)

(72) Inventors: Paul SMITH, Smithfield, RI (US); Rahul PRABHU, Brighton, MA (US); Lucas FRANKLE, Huntingtown, MD (US); Jonathan ROOT, Townsend, MA (US)

(73) Assignee: Boston Scientific Scimed, Inc., Maple Grove, MN (US)

(21) Appl. No.: 19/039,132

(22) Filed: Jan. 28, 2025

Related U.S. Application Data
(60) Provisional application No. 63/627,138, filed on Jan. 31, 2024.

(51) Int. Cl. A61F 13/05; A61B 17/00; A61F 13/20

(52) U.S. Cl. CPC A61F 13/05; A61B 17/00234; A61F 13/2045; A61B 2017/00292; A61B 2217/005; A61F 2013/2014

(57) ABSTRACT

A distal end of a medical device may comprise a tube configured to be coupled to a source of suction; and a porous body coupled to the tube. The porous body may have a wall with an inner surface and an outer surface. The distal end may include a cavity, such that a gap extends from an outer surface of the tube to the inner surface of the porous body.

### CROSS-REFERENCE TO RELATED APPLICATIONS

This application claims the benefit of priority to U.S. Provisional Application No. 63/627, 138, filed on Jan. 31, 2024, which is incorporated by reference herein in its entirety.

### TECHNICAL FIELD

Various aspects of this disclosure relate generally to minimally invasive medical devices and methods. In particular, aspects of the disclosure relate to medical devices and methods for endoscopic medical procedures, such as closing a wound or otherwise treating tissue.

### BACKGROUND

Endoscopic and open surgical procedures of the gastrointestinal (GI) tract include, for example, colonic resection, bariatric surgery, esophagostomy, gastric bypass, and sleeve gastrectomy, among others. These procedures may result in perforation, post-surgical anastomotic leaks, or other wounds of the GI tract. Patients with perforations, post-surgical anastomotic leaks, and/or other wounds in the GI tract have high mortality rates with limited treatment options. Options include endoscopic placement of clips or stents, endoscopic sutures or sealants, or surgical re-operation. Surgery is relatively invasive and has high morbidity and mortality rates. While endoscopic stent placement is a less invasive option, the stent can migrate from the intended location and/or wall off infection at a treatment site, inhibiting drainage.

The medical devices and methods of the current disclosure may rectify some of the deficiencies described above or address other aspects of the art.

### SUMMARY

Each of the aspects disclosed herein may include one or more aspects of the features described in connection with any of the other disclosed aspects.

Aspects of the disclosure relate to, among other things, a medical device for Endoluminal vacuum therapy and related methods. These aspects may include one or more of the features described below.

For example, the disclosure includes a distal end of a medical device comprising a tube configured to be coupled to a source of suction; and a porous body coupled to the tube. The porous body may have a wall with an inner surface and an outer surface. The distal end may include a cavity, such that a gap may extend from an outer surface of the tube to the inner surface of the porous body.

According to some aspects, the distal end may include a cap positioned in the cavity. The cap may comprise a porous material. A proximal surface of the cap may be a distalmost end of the cavity. The distal end may include a rib extending from an outer surface of the tube to the inner surface of the porous body. The distal end may include a second rib extending from the outer surface of the tube to the inner surface of the porous body. The rib may define a first portion of the cavity that is proximal of the rib and a second portion of the cavity that is distal to the rib. A distal portion of the porous body may be coupled to a distal portion of the tube. In a relaxed configuration, the porous body may taper radially outward in a proximal direction from a location where the porous body is coupled to the tube. The porous body may have a bell shape in a relaxed configuration. The porous body may have a tubular shape from a first axial location of the porous body to a second axial location of the porous body. The cavity may have a uniform radial width from the first axial location to the second axial location. The porous body may have a prismatic shape from a first axial location to a second axial location. The porous body may have a plurality of ridges and a plurality of troughs. In some aspects, the distal end may be configured such that when the distal end is positioned in a body of a patient and the tube is connected to the source of suction, bodily fluids flow through pores of the porous body, through the cavity, and into the tube.

The disclosure also includes a distal end of a medical device comprising a tube configured to be coupled to a source of suction; and a porous body coupled to the tube. The distal end may include a cavity between the porous body and the tube. A cap may be positioned at a distal portion of the porous body, such that a proximal surface of the cap is a distalmost end of the cavity.

According to some aspects, the cap may comprise porous material. The cavity may be configured to receive material from an environment external to the porous body through pores of the porous body.

The disclosure also includes a distal end of a medical device comprising a tube configured to be coupled to a source of suction; and a porous body coupled to the tube. The distal end may include a cavity between the porous body and the tube. A rib may extend from an outer surface of the tube to an inner surface of the porous body, such that the rib defines a first portion of the cavity that is proximal of the rib and a second portion of the cavity that is distal to the rib. According to some aspects, the distal end may further comprise at least a second rib extending from an outer surface of the tube to the inner surface of the porous body.

### BRIEF DESCRIPTION OF THE DRAWINGS

The accompanying drawings, which are incorporated in and constitute a part of this specification, illustrate various aspects of this disclosure and together with the description, serve to explain the principles of the disclosure.

FIGS. 1A-1C depict aspects of an exemplary medical device, in accordance with some aspects of the disclosure.

FIG. 2 depicts a cross-sectional view of another exemplary medical device, in accordance with some aspects of the disclosure.

FIG. 3 depicts a cross-sectional view of another exemplary medical device, in accordance with some aspects of the disclosure.

FIGS. 4A-4B depict cross-sectional views of further exemplary medical devices, in accordance with some aspects of the disclosure.

FIGS. 5A-5B depict aspects of another exemplary medical device, in accordance with some aspects of the disclosure.

### DETAILED DESCRIPTION

Particular aspects of the disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body.

As used herein, the terms “comprises,” “comprising,” “including,” “includes,” “having,” “has” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.”

Further, relative terms such as, for example, “about,” “substantially,” “approximately,” etc., are used to indicate a possible variation of ±10% in a stated numeric value or range.

Endoluminal vacuum therapy (EVT or EVAC, and referred to herein as EVAC) is a procedure to treat wounds, such as post-surgical leaks or perforations in the gastrointestinal tract (GI) following a surgical or endoscopic procedure, such as colonic resection, bariatric surgery, or esophagostomy. In EVAC, negative pressure is delivered to the wound site in a body of a patient (e.g., in the GI tract) through a nasogastric tube having a porous body, such as a sponge or sponge-like material or foam (e.g., vacuum sealed foam) sutured at its distal end. A proximal end of the tube may be connected to a collection container. The porous body may be placed endoscopically into the perforation, leak, or wound. In some examples, EVAC includes endoluminal placement of a foam or other like material into the wound (e.g., target site or wound cavity), including a perforation, a leak, a cyst, an anastomosis, etc. Placement of the porous body may be via a catheter, scope (endoscope, bronchoscope, colonoscope, duodenoscope, gastroscope, etc.), tube, or sheath, inserted into the GI tract via a natural orifice. The orifice can be, for example, the nose, mouth, or anus, and the placement can be in any portion of the GI tract, including the esophagus, stomach, duodenum, large intestine, or small intestine. Placement of the porous body may also be in other organs reachable via the GI tract (e.g., the colon).

Rat-tooth forceps or another accessory device may be extended through a working channel of the scope and used to guide the foam to the wound site as the scope is navigated to the wound site. Negative pressure is then applied. The porous body in the wound, along with the negative pressure, may accelerate healing by encouraging local tissue granulation at the wound site. The porous body may be replaced with increasing smaller sizes of porous body as the wound heals and closes.

Present devices suited for EVAC are limited. For example, the tortuous and often narrow configuration of the GI space may make delivery of conventional porous bodies to the target site difficult. Conventional porous bodies may lack compressibility, which limits navigation of a delivery device through narrow body lumens/openings. In addition, fluid material commonly encountered in EVAC may be thick and may form clumps of biological material within the tortuous pore structure of the porous body, resulting in clogging or reduced suction.

Features of the medical devices and methods herein may improve suction, delivery of a porous body to a target site, and/or provide a porous body with varying properties and/or characteristics suitable for treatment of wound sites or wound cavities of varying sizes. Additionally, components of the medical devices described herein may be packaged as a kit. The features of the below devices may be combined with one another in any combination or subcombination.

According to some aspects of the disclosure, the medical devices may include a distal portion having a porous body (e.g., a foam or a sponge) coupled to a distal portion of a tube. The porous body may have an outer wall that surrounds a distal portion of the tube. The distal portion of the medical device may include a cavity (e.g., an empty or hollow space) disposed between the outer wall of the foam and the tube. For example, the porous body may include a hollowed-out portion that forms the cavity in which the tube is received. The distal portion of the medical device may thus include a foam shell while providing for a gap between the tube and the porous body. The outer wall of the porous body may radially surround the cavity. The cavity may vary in diameter/width and the outer wall may vary in thickness depending on, for example, the size of the wound.

As compared with conventional distal portions of EVAC devices, the disclosed distal portions may have an overall similar outer diameter, while including a reduced thickness of the porous body (e.g., foam). The cavity may allow for improved suction functionality, reducing the risk of forming clumps in the porous body. Additionally, the cavity may provide a decreased total volume of the porous body, allowing for increased compressibility the porous body.

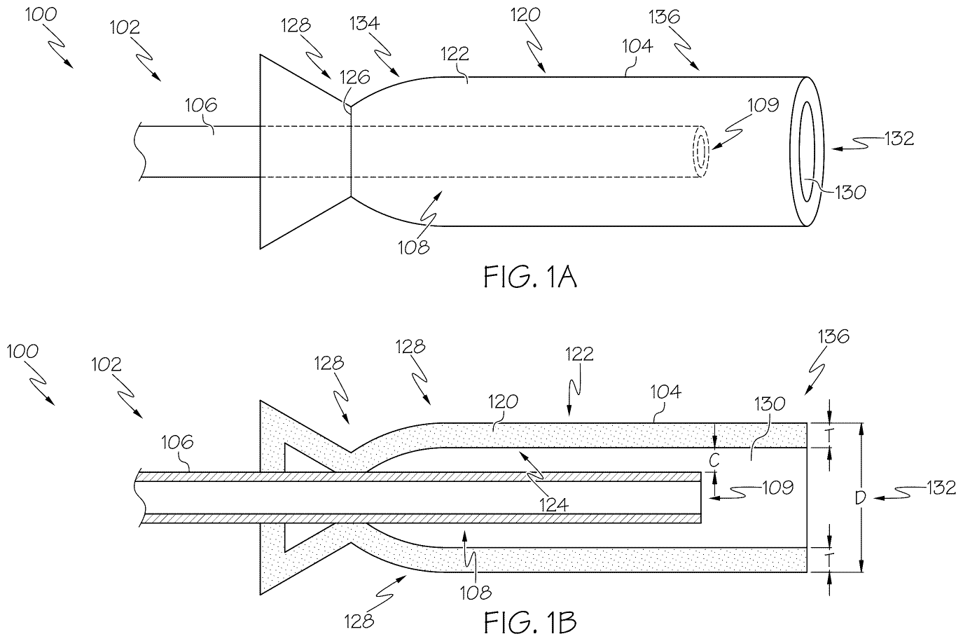

FIGS. 1A-1C depict an exemplary medical device 100, which may be used for EVAC procedures. FIG. 1A shows a perspective view of the medical device 100. FIG. 1B shows a cross-sectional view of the medical device 100 taken along a central longitudinal axis of the medical device 100. FIG. 1C shows a cross-sectional view of the medical device 100 taken perpendicularly to the central longitudinal axis of the medical device 100. The medical device 100 may include a distal end 102. The distal end 102 may include a porous body 104 (e.g., a foam, sponge, or other cellular structure), which may be coupled to the distal portion 108 of a tube 106.

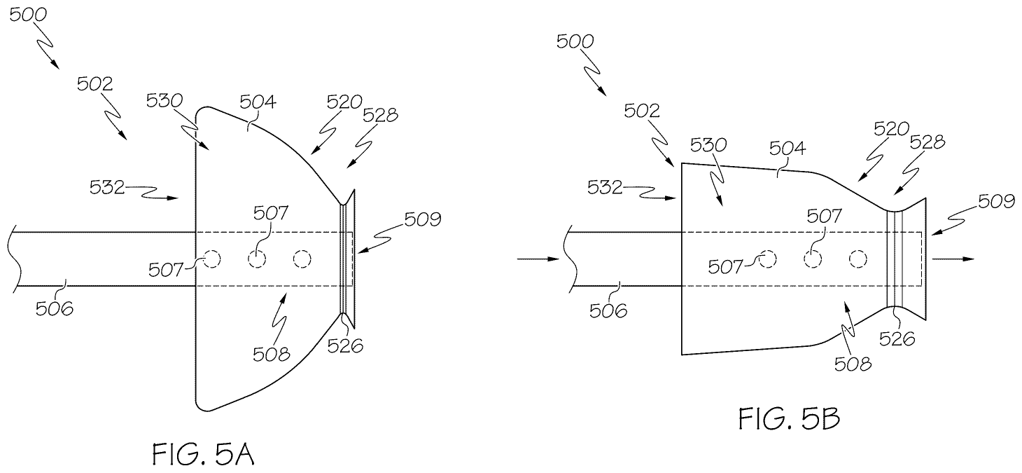

The tube 106 may be a vacuum tube, which may include a lumen for transmitting suction, to provide aspiration and removal of unwanted materials from the target site. The tube 106 may be composed of a material compatible with medical applications, and that provides a degree of flexibility. The tube 106 may extend from a proximal end of the tube (not shown) to the distal portion 108 of the tube 106. A proximal end of the tube may be connected to a vacuum or suction source (not shown and having any of the properties of a vacuum or suction source known in the art), which may supply a negative pressure to the porous body 104. The negative pressure may pull fluid, material, and/or other debris into the tube 106, such that material drawn into the distal portion 108 of the tube 106 may flow proximally. The distal portion 108 of the tube 106 may have a distal opening 109 and one or more side openings (not shown but similar to side openings 507 shown in FIGS. 5A-5B) on a radial outer wall of the tube 106.

The distal end 102 may include a porous body 104 (e.g., a foam, sponge, or other cellular structure), which may be coupled to the distal portion 108 of tube 106. The porous body 104 may have an outer wall 120. The wall 120 may have an approximately tubular shape, such that the wall 120 has an approximately annular cross-sectional shape, although such a shape is merely exemplary. The wall 120 may have an outer surface 122 (e.g., a radially outer surface) and an inner surface 124 (e.g., a radially inner surface). As shown in FIGS. 1B and 1C, the wall 120 may have a thickness or width “T” between the outer surface 122 and the inner surface 124. Thickness “T” may be measured in a direction perpendicular to a central longitudinal axis of the tube 106 (e.g., a radial direction). The thickness or width of the wall “T” may be consistent and uniform throughout the porous body 104. Alternatively, the thickness “T” may vary along a length and/or circumference of the porous body 104. The porous body 104 may be any suitable size or thickness; the size or thickness “T” of the porous body 104 may be selected based on a location of treatment within the anatomy, properties of a wound to be treated, a stage of treatment, or any other relevant factors.

The porous body 104, including the wall 120, may include any suitable material, such as sponges, foams, or other porous structures that facilitate suction and mitigate the risk of clumping or obstruction. For example, suitable materials may include polyvinyl alcohol (PVA), polyurethane, polyester urethane, polyether urethane, silicone rubber or similar. A plurality of pores may extend through the porous body 104. The pores may form pathways from the outer surface 122 to the inner surface 124 through the wall 120. Within the porous body 104, the pores may create a network of interconnected pathways. These pathways may serve as channels for the movement of fluids, gases, or other materials. The foam structure may incorporate a variety of pore sizes and densities. For example, areas with larger pores might facilitate the suction of larger debris, while smaller pores may prevent clumping.

The porous body 104 may be coupled to the tube 106 via, for example, a fastener 126. The fastener 126 may be a suture or any other suitable fastening mechanism known in the art (e.g., staple, tie, crimp, etc.) The fastener may be positioned on the porous body 104, for example, in a proximal portion 128 of the porous body 104 and/or near the proximal-most end of porous body 104. As shown in FIGS. 1A and 1B, the fastener 126 may be tightened such that the inner surface 124 may abut the tube 106 at a location of the fastener 126. The proximal portion 128 of the porous body 104 proximal to the fastener 126 may flare outward, forming a skirted shape. Alternatively, and as described below, the fastener 126 may be moveable and/or positioned in various sites of the porous body 104. For example, the fastener 126 may be positioned at a distal portion of the porous body 104 as shown in FIGS. 5A-5B. A distalmost end of the tube 106 may be proximal of a distalmost end of the porous body 104, level with a distalmost end of the porous body 104, or distal to a distalmost end of the porous body 104.

The distal end 102 may include a lumen or the cavity 130 that extends between the tube 106 and the inner surface 124 of wall 120 of the porous body 104. Formed between the tube 106 and the wall 120, the cavity 130 may be a gap or empty space. By having a cavity 130 between the tube 106 and the wall 120 of the porous body 104, the tube 106 may be out of contact with the porous body 104 distal to fastener 126/proximal portion 128. In other words, an open space may extend between the tube 106 and the wall 120 to form the cavity 130. In some examples, the cavity 130 may form a gap between the tube 106 and the wall 120 along an entire length of the wall 120 that is distal of the fastener 126/proximal portion 128. As discussed below, the cavity 130 contributes, among other things, to the overall compressibility of the porous body 104. This compressibility is advantageous for navigating through narrow body lumens or openings, providing flexibility during an EVAC procedure. Additionally, the presence of the cavity 130 contributes to improved suction functionality. The cavity 130 may allow for a more direct and unobstructed path for the suction of fluids or materials through the tube 106, minimizing the risk of clogging or impediments to the suction process.

As shown in FIGS. 1B and 1C, the cavity 130 may have a width “C.” The width “C” may extend in the same direction as thickness “T” of the wall 120 described above. The width “C” may be perpendicular to the central longitudinal axis of the tube 106 (e.g., in a radial direction). The width “C” may be measured from the inner surface 124 to the outer surface of the tube 106. In the embodiment, the width “C” of the cavity 130 is wider than the thickness “T” of the wall 120. However, the thickness “T” of the wall 120 may be wider than the width “C” of the cavity 130. In some examples, the width “C” may be approximately equal to a diameter of the tube 106.

The cavity 130 may extend from the position of the fastener 126 to a distalmost end of the porous body 104. The cavity 130 may provide a continuous channel along an entire length of the porous body 104 distal to the fastener 126. In some examples, the cavity 130 may have a uniform width “C” along at least a portion of an axial length of cavity “C”. For example, the cavity 130 may have an approximately uniform width “C” from a middle portion 134 (first axial location) of the distal end 102 to a distal portion 136 (second axial location) of the distal end 102 (see FIG. 1B). A portion of the wall 120 that is proximal of the middle portion 134 of the distal end 102 may taper inward in a proximal direction, toward the fastener 126. Thus, a portion of the cavity 130 that is proximal of the middle portion 134 may have a width “C” that tapers inward in a proximal direction toward the fastener 126. The cavity 130 may have a distal opening 132, such that the cavity 130 is open to the outside environment on the distal portion 108 of tube 106.

As shown particularly in FIG. 1C, the cavity 130 may have an annular cross-sectional shape. The distal opening 132 may also have an annular shape. A radially inner side of the cavity 130 may have a shape that corresponds to a shape of an outer surface of the tube 106. A radially outer side of the cavity 130 may have a shape corresponding to a shape of the inner surface 124 of the wall 120 of the porous body 104.

As shown particularly in FIG. 1C, the medical device 100 may have an overall radius of “D.” The radius “D” may be half of a diameter/width of the medical device 100. The term “radius” is not limited herein to refer only to tubular devices with circular cross-sections and includes half of the overall width of devices with non-circular cross-sections. The width “D” of the medical device 100 may be equal to a radius of the tube 106 plus width “C” plus thickness “T.” Compared to the distal ends of EVAC devices lacking the cavity 130 and having the same overall width or radius “D” (i.e., EVAC devices featuring a porous body that is directly adjacent to a vacuum tube), the porous body 104 occupies less total volume, radial thickness, and/or width of the distal end 102. In other words, the distal end 102 has more empty space (in the form of cavity 130) as compared with other distal ends of EVAC devices. It will be appreciated that thickness “T,” width “C,” and radius “D” may have any suitable values, which may be constant along an entire axial length of the medical device 100 or may vary along the length of the medical device 100.

In some examples and as discussed above, the porous body 104 may have a tubular shape cinched by the fastener 126 and flaring outward proximally and distally from the fastener 126. For example, the porous body may have a cone shape (which may taper in a proximal or distal direction). In these examples, the cavity 130 may form because the inner diameter of the wall 120 (extending between opposing sides of the inner surface 124) is larger than the outer diameter of the tube 106. When not positioned in a wound, the porous body 104 may have a tubular shape from the middle portion 134 of the distal end 102 to the distal portion 136 of the distal end 102. The cavity 130 may have a uniform radial width from the middle portion 134 of the distal end 102 to a distal portion 136 of the distal end 102. The porous body 104 may also have alternative shapes suitable for certain wound types, shapes of wounds, or anatomical features. When positioned at or within a wound in a body of a patient, body tissue may act on the porous body 104 to alter the shape shown in the Figures and described above.

Additionally or alternatively, the cavity 130 within the porous body 104 may be a carved-out space, leading to a decreased total volume of the porous body 104 while maintaining the overall radius “D” of the porous body 104. For example, a user may form the cavity 130 by removing material from the porous body 104, leaving a hollowed-out region corresponding to the cavity 130. A user may select the dimensions and shape of the cavity 130 based on user preferences or properties of a wound. Alternatively, the porous body 104 may be pre-molded to form the cavity 130.

The medical device 100 (particularly the distal end 102) reduces the risk of clogging and increases suction due to the presence of the cavity 130. During a medical procedure (e.g., EVAC), in which the distal end 102 is positioned in a body of a patient, the distal end 102 receives material from the EVAC site (which is an environment external to the porous body) through the thickness “T” of the wall 120 of the porous body 104, which is the distance between the outer surface 122 and the inner surface 124. The material then travels through the width “C” of the cavity 130. Thus, a portion of the distance that the material travels is through open space (the cavity 130), rather than through the porous body 104. This reduces the risk of clumping or obstruction of the distal end 102 and improves suction. The reduced clogging risk contributes to sustained negative pressure at the EVAC site, promoting tissue granulation, and may accelerate the healing process at the wound site.

The presence of the cavity 130 within the distal end 102 of the medical device 100 increases compressibility (e.g., radial compressibility) of the distal end 102. The compressibility of the distal end 102 may facilitate the insertion of the medical device 100 into a tubular or other delivery device (e.g., smaller delivery devices than distal ends 102 without a cavity 130). The distal end 102 may also be compressed so as to pass through working channels of endoscopes or other similar introduction devices.

A configuration of device 100 may allow for a diameter of the tube 106 (including an inner diameter of tube 106) and the distal opening 109 of the tube 106 to be increased. A larger inner diameter enables higher fluid flow volumes through the tube 106. A larger inner diameter of the tube 106 minimizes the risk of material clumping or clogging within the tube 106 as suction capacity increases.

The configuration of the medical device 100 maintains an outer surface area of porous body 104, which provides a high contact surface area with surrounding tissue and a distribution of suction force. As tissue is pulled into the area, the cavity 130 may collapse, allowing tissue growth and healing. An outer diameter of the porous body 104 may be increased relative to other EVAC devices due to the compressibility provided by the cavity 130. Furthermore, the porous body 104 may have a longer axial length relative to other types of EVAC devices, while retaining compressibility of the distal end 102 of the medical device 100. Additionally, the open space within the cavity 130 may allow for the passage of guidewires or other medical accessories through the cavity, enabling physicians to navigate through anatomical structures with greater ease (e.g., by pushing tissue out of the way or allowing navigation over a guidewire).

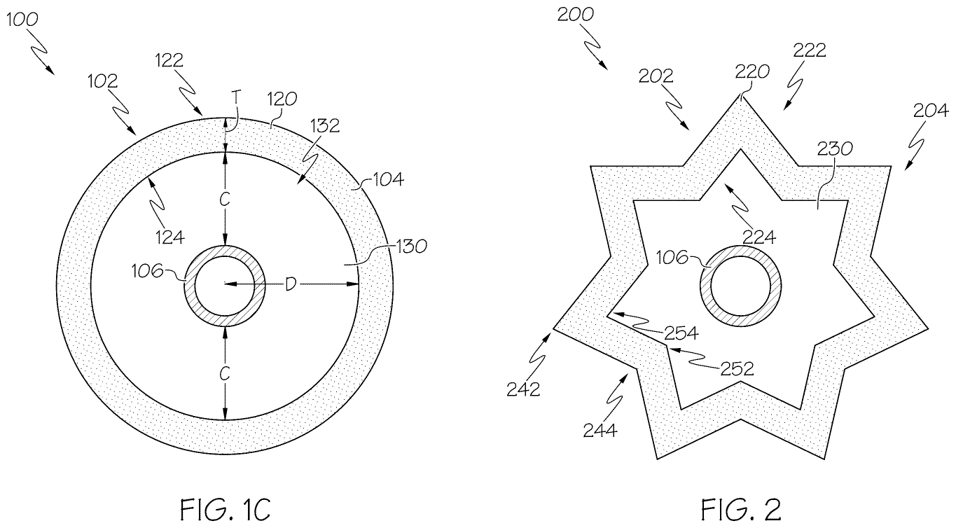

FIG. 2 shows a cross-sectional view of an exemplary alternative distal end 202 of a medical device 200. The cross-sectional view of FIG. 2 is taken perpendicularly to a longitudinal axis of the tube 106. The medical device 200 may have any of the properties of medical device 100 unless otherwise specified herein. Whereas the porous body 104 of the medical device 100 may have an approximately annular cross-section, a porous body 204 of the medical device 200 may have a prismatic shape with a polygonal cross-section. For example, the porous body 204 may have a prismatic shape from a first axial location to a second axial location. As used herein, the term “prismatic” refers to a shape of the wall 220 and does not require end faces of a prism. In other words, body 204 may have a plurality of alternating V-shaped channels and a plurality of V-shaped ridges. Stated differently, wall 220 may include a plurality of portions having a chevron-shaped cross-section. As with the medical device 100, a cavity 230 extends between the tube 106 and the porous body 104. The cavity 230 may have any of the properties of the cavity 130.

FIG. 2 shows a wall 220 of the porous body 204 as having a cross-sectional 7-pointed star shape (i.e., as having 14 sides). However, such a shape is merely exemplary, and the porous body 204 may have any cross-sectional shape. An inner surface 224 of the wall 220 may be similar to an outer surface 222 of the wall 220 and include the same shape but different sizes. The inner surface 224 and the outer surface 222 of the wall 220 may have comparable geometric configurations. For instance, the inner surface 224 may mirror the particular curvature, contour, or pattern of the outer surface. Alternatively, the inner surface 224 and the outer surface 222 may have different shapes.

The outer surface 222 may have a plurality of ridges 242 and a plurality of troughs 244. The ridges 242 may be raised or elevated portions of the outer surface 222 (radially outermost portions of the outer surface 222). In other words, the ridges 242 may protrude radially outwardly from the outer surface 222. The troughs 244 may be depressions or grooves in the outer surface 222 (radially innermost portions of the outer surface 222). The inner surface 224 may also have a plurality of ridges 252 and troughs 254. Ridges 252 of the inner surface 224 may protrude radially inward (e.g., radially innermost portions of the inner surface 224) and the troughs 254 may be radially outermost portions of the inner surface 224. The ridges 242, 252 may alternate with the troughs 244, 254, respectively. A first radius extending through the porous body 204 may extend through one of the ridges 242 and one of the troughs 254. A second radius extending through the porous body may extend through one of the troughs 244 and one of the ridges 252. The ridges 242, 252, and the troughs 244, 254 may give the porous body 204 a pleated shape.

The ridges 242 and 252 and the troughs 244 and 254 may extend parallel to a longitudinal axis of medical device 200. Each of the ridges 242, 252, and the troughs 244, 254 may be parallel to all of the others of the ridges 242, 252, and the troughs 244, 254. The ridges 242, 252 and the troughs 244, 254 may facilitate collapsing the porous body 204 to deliver the porous body 204 to a target body lumen or cavity. For example, the troughs 244 may create predetermined points of flexibility in the structure of the porous body 204 to facilitate collapsing porous body 204 radially inward. In other words, porous body 204 may collapse radially inward along pleats of porous body 204.

The cavity 230 extends from the inner surface 224 to tube 106. The shape of an outer edge of cavity 230 may also be prismatic, with the outer edge of cavity 230 polygonal cross-section corresponding to the shape of the inner surface 224. An inner edge of cavity 230 may have a circular cross-section, corresponding to an outer surface of the tube 106. However, the shape shown and described with respect to FIG. 2 is merely exemplary. The cavity 230 may have any shape in accordance with the shapes of porous body 204 and tube 106.

The shapes of porous bodies 104, 204 are merely exemplary, and the porous bodies 104, 204 may have any suitable shape. For example, the porous body 204 may have any prismatic or non-prismatic (e.g., irregular) shape. Irregular shapes may be tailored to specific anatomical features or wound sizes, providing a more customized solution for diverse medical situations. A cross-section of the porous bodies 104, 204 may also have any suitable shape. For example, a cross section of the porous bodies 104, 204 may be a regular polygon with uniform sides and angles, such as a triangle, square, or hexagon. Alternatively, cross-sections of the porous bodies 104, 204 may be irregular polygons having sides and angles of varying lengths and measures. The cross-sections of the porous bodies 104, 204 may be concave polygons (as shown for porous body 204) or convex polygons.

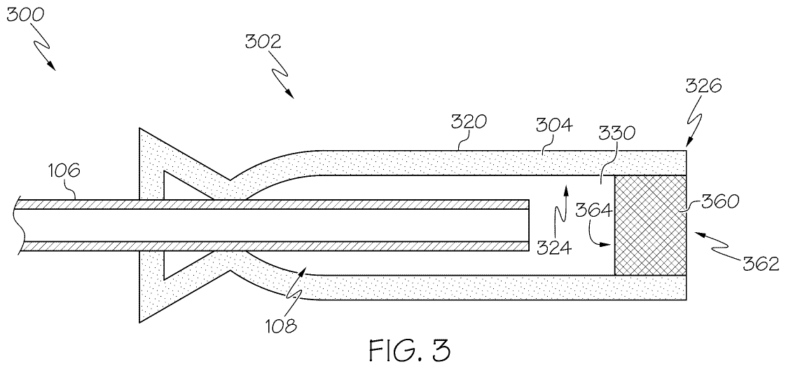

FIG. 3 depicts a distal end 302 of an alternative medical device 300, which may have the properties of the medical devices 100, 200, discussed above, except as specified herein. FIG. 3 is a cross-sectional view taken along a central longitudinal axis of the medical device 300. The medical device 300 may include a porous body 304, including any of the properties of the porous bodies 104 and 204, disposed around the tube 106. The medical device 300 may include a cavity 330, including any of the properties of the cavity 130 and the cavity 230. The medical device 300 may further include a cap 360 at a distalmost end of the porous body 304, which is described in further detail below.

The cap 360 may be positioned within the porous body 304 at a distalmost end of the porous body 304. The cap 360 may be formed from the same porous material as the porous body 304 (e.g., polyvinyl alcohol (PVA), polyurethane, polyester urethane, polyether urethane, silicone rubber or similar). Alternatively, the cap 360 may be composed of a non-porous material or of a different porous material than the porous body 304.

The cap 360 may be integrally formed with the porous body 304, from a same piece of material as porous body 304. Alternatively, the cap 360 may be a separate piece of material that is coupled to the porous body 304. For example, the cap 360 may be retained within the porous body 304 via a frictional fit, adhesive, sutures, or other mechanisms. In some examples, the cap 360 may be included in a kit with other portions of the medical device 300. The user may selectively use or integrate the cap 360 with the components of medical device 300. Alternatively, the medical device 300 may be packaged with the cap 360 and affixed to the porous body 304.

As shown in FIG. 3, the cap 360 may be received within the cavity 330, so a radially outer surface of the cap 360 abuts an inner surface 324 of a wall 320 of the porous body 304. In one aspect, a distalmost end 362 of the cap 360 may be axially aligned with a distalmost end 326 of the porous body 304. Alternatively, the distalmost end 362 of the cap 360 may extend beyond the distalmost end 326 of the porous body 304 in a distal direction. The cap 360 may protrude or extend beyond the porous body in this configuration. Alternatively, the distalmost end 362 of the cap 360 may be proximal of the distalmost end 326 of the porous body 304. Alternatively, the cap 360 may be fixed to a distalmost end of the porous body 304, such that a proximalmost end of the cap 360 abuts a distalmost end of the wall 320.

The cavity 330 may have a distal end aligning with or reaching a proximal surface 364 of the cap 360. The tube 106 may have a distalmost end that is proximal of a proximal end of the cap 360 or contacting the cap 360. Alternatively, the tube 106 may extend into the cap 360.

The cap 360 may have any suitable shape. For example, as shown in FIG. 3, the cap 360 may have a cylindrical shape. A distal surface of the cap 360 may be rounded. The cap 360 may possess a radially outer surface complementarily shaped to the inner surface 324 of the wall 320. The cap 360 may be of any suitable axial length.

The cap 360 may prevent the free flow of material into the cavity 330 through a distal end of the porous body 304. The cap 360 may, therefore, allow for a more controlled fluid ingress into the tube 106 during EVAC. The cap 360 may also act as a barrier, reducing the risk of contamination by preventing undesired materials or substances from entering the cavity 330.

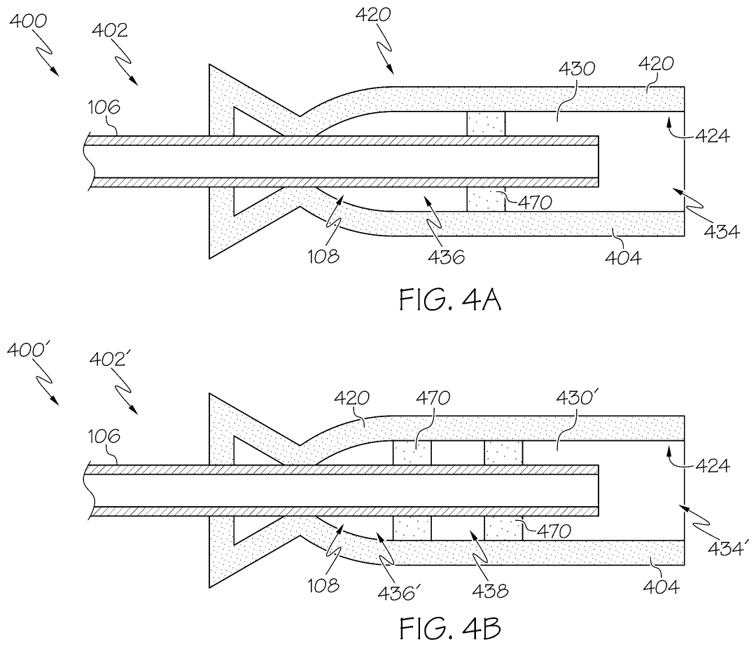

FIGS. 4A-4B depict cross-sectional views of the distal ends 402, 402′ and the alternative medical devices 400, 400′, taken along respective central longitudinal axes of the medical devices 400, 400′. The medical devices 400 and 400′ may have any of the same features except as specified below. Furthermore, the medical devices 400 and 400′ may have any of the features of the medical devices 100, 200, or 300 described above, except where specified.

As shown in FIG. 4A, a distal end 402 of the medical device 400 may include the distal portion 108 of the tube 106 and a porous body 404. The porous body 404 may have any features of the porous bodies 104, 204, and 304. The distal end 402 may include at least one rib 470 that may support the porous body 404.

The rib 470 may extend between the outer surface of the tube 106 and an inner surface 424 of a wall 420 of the porous body 404. The rib 470 may be integrally formed with porous body 404 (formed of a single piece of material). Alternatively, the rib 470 may be separate from the porous body 404. In this aspect, the rib 470 may be coupled to the porous body 404 using any suitable mechanism (e.g., adhesive, sutures, other mechanical structures, friction fit). The rib 470 may be formed of any material as the cap 360.

In one aspect, the rib 470 may extend around the entire circumference or perimeter of the tube 106 and the inner surface 424. Therefore, the rib 470 may be continuous and completely encircle tube 106 and the inner surface 424. In such an example, the rib 470 may be washer shaped, with a flat and generally circular configuration with a central opening. The washer shape is merely exemplary, and the rib 470 may be any suitable shape. Alternatively, the rib 470 may extend around only a portion of the circumference or perimeter of the tube 106 and/or the inner surface 424. In such examples, more than one rib 470 may be positioned at the same axial position along distal end 402, with each rib extending partially around a circumference of tube 106/inner surface 424.

The cavity 430 may have any of the features of the cavity 130, 230, and 330 described above, except where specified. The rib 470 extends along only a partial longitudinal length of the porous body 404, resulting in a cavity 430 extending between the porous body 404 and the tube 106 at portions of the distal end 402 not having the rib 470. The cavity 430 may be subdivided into a plurality of portions/segments, for example, a distal portion 434 and a proximal portion 436. In some examples, the rib 470 may include openings to facilitate fluid communication between the distal portion 434 and the proximal portion 436, in addition to the fluid communication provided by the pores of the porous body 404 and the pores of rib 470. The rib 470 may be provided as a modular component, included as part of a user-installable kit. The user may install the rib 470 onto the porous body 404 during assembly. Alternatively, the rib 470 may already be installed on the porous body 404 upon delivery of the medical device 400.

As shown in FIG. 4B, the medical device 400′ may have a distal end 402′ that has any of the properties of the distal end 402, except that the distal end 402′ may include a plurality of the rib(s) 470. For example, as shown in FIG. 4B, the distal end 402′ may include at least two rib(s) 470. The rib(s) 470 may be evenly or unevenly spaced along a longitudinal length of the porous body 404. In an embodiment, the rib(s) 470 may divide or define a cavity 430′ into three portions: the distal portion 434′, the proximal portion 436′, and the middle portion 438. In further embodiments, the additional rib(s) 470 may be configured to further subdivide the medical device 400′.

The rib(s) 470 may provide structural support by increasing radial force outward to the porous body 404s. For example, the rib(s) may help prevent collapse or deformation of the porous body 404 and may keep the cavities 430, 430′ open. Additionally, depending on the orientation and the design of rib(s) 470, the flow of fluids or materials within the porous body 404 and the cavity 430 may be controlled or adjusted. The rib(s) 470 may section off pockets of open space within the cavity 430, which may be helpful if a section (e.g., the proximal portion 436′) is clogged and ensures other sections remain unaffected during EVAC.

FIGS. 5A-5B shows a distal end 502 of another exemplary medical device 500 in a relaxed configuration (FIG. 5A) and in an insertion configuration (FIG. 5B). The medical device 500 may have any of the features of the medical devices 100, 200, 300, 400, 400′, unless otherwise specified, and any of the features above (e.g., rib(s) 470, cap 360) may be used in conjunction with the medical device 500. Whereas medical device 100 may be fixed to the tube 106 at a proximal portion of the porous body 104, a porous body 504 of the medical device 500 may be coupled to a tube 506 at or near a distalmost end of a porous body 504.

The porous body 504 may include a wall 520. The wall 520 may be similar to for example, the wall 120, and may have an annular cross-sectional shape, although such a shape is exemplary. The wall 520 may have any of the shapes discussed above for any of the previous examples. In a natural, relaxed state (FIG. 5A), the porous body 504 may have a narrower distal end near a fastener 526 that couples the porous body 504 to the tube 506, and a wider proximal end. The shape of the wall 520 of the porous body 504 may taper radially outward moving in a proximal direction from the fastener 526. For example, the porous body 504 may have a bell shape.

The porous body 504 may be coupled to the tube 506 at or near a distalmost end of the porous body 504 and/or the tube 506. The fastener 526 may be a suture or any other suitable fastening mechanism known in the art (e.g., staple, tie, crimp, etc.) The fastener 526 may have any of the properties of the fastener 126. The fastener 526 may be positioned on the porous body 504, for example, at a distal portion 528 of the porous body 504 and/or near the distalmost end of the porous body 504. A portion of the porous body 504 distal to the fastener 526 may flare outward, forming a skirted shape. A distalmost end of the tube 506 may be proximal, distal, or axially level with a distalmost end of the porous body 504.

The distal end 502 may include a lumen or the cavity 530 that extends between the wall 520 and the tube 506 at portions proximal of the fastener 526. Formed between the tube 106 and the wall 520, the cavity 530 may be a gap or space and share any of the properties of the cavities 130, 230, 330, 430, and 430′. The cavity 530 may extend from the position of the fastener 526 to a proximalmost end of the porous body 504. The cavity 530 may provide a continuous channel throughout the porous body 504, proximal of the fastener 526. The cavity 530 may have an aperture or an opening 532, such that the cavity 530 is open to the outside environment on a proximal end of the porous body 504. Alternatively, the distal end 502 may have a cap like the cap 360. The cap (e.g., cap 360) may be positioned within the porous body 504 at a proximalmost end of the porous body 504 to for example, prevent the free flow of material into a proximal end of the cavity 530. The distal end 502 may include an optional distal opening at distal end 509.

The tube 506 may have a closed (e.g., plugged) distal end 509 and openings 507 along a sidewall of the tube 506. Thus, bodily fluids and other materials may flow into a central lumen of the tube 506 via the openings 507. The openings 507 may be distal of a proximalmost end of the porous body 504, such that bodily fluids and materials may first flow through the porous body 504 before entering the openings 507.

As the distal end 502 of the medical device 500 is moved distally (a direction shown by the arrow in FIG. 5B) through a body (e.g., dragged distally by a forceps or other device), the porous body 504 may compress radially inward due to forces exerted by a body lumen on porous body 504. In other words, a radial thickness of the porous body 504 may decrease during the insertion of the distal end 502. Thus, the medical device 500 may help to prevent bunching of the porous body 504 during insertion of the medical device 500. When the distal end 502 is positioned at a treatment location, the distal end 502 may revert to its relaxed, bell shape (FIG. 5A).

Thus, as compared with devices in which a porous body is coupled at a proximal end of the porous body, properties of the medical device 500 may help to ease insertion of the medical device 500. Furthermore, the medical device 500 may be easier to load in a delivery device. For example, the distal end 502 may be forward-loaded into a delivery device/carrier, rather than back-loaded.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. A distal end of a medical device comprising:
a tube configured to be coupled to a source of suction; and
a porous body coupled to the tube, wherein the porous body has a wall with an inner surface and an outer surface;
wherein the distal end includes a cavity, such that a gap extends from an outer surface of the tube to the inner surface of the porous body.

2. The distal end of claim 1, wherein a cap is positioned in the cavity.

3. The distal end of claim 2, wherein the cap comprises a porous material.

4. The distal end of claim 2, wherein a proximal surface of the cap is a distalmost end of the cavity.

5. The distal end of claim 1, further comprising a rib extending from an outer surface of the tube to the inner surface of the porous body.

6. The distal end of claim 5, further comprising at least a second rib extending from the outer surface of the tube to the inner surface of the porous body.

7. The distal end of claim 5, wherein the rib defines a first portion of the cavity that is proximal of the rib and a second portion of the cavity that is distal to the rib.

8. The distal end of claim 5, wherein a distal portion of the porous body is coupled to a distal portion of the tube.

9. The distal end of claim 8, wherein, in a relaxed configuration, the porous body tapers radially outward in a proximal direction from a location where the porous body is coupled to the tube.

10. The distal end of claim 8, wherein the porous body has a bell shape in a relaxed configuration.

11. The distal end of claim 1, wherein the porous body has a tubular shape from a first axial location of the porous body to a second axial location of the porous body.

12. The distal end of claim 11, wherein the cavity has a uniform radial width from the first axial location to the second axial location.

13. The distal end of claim 1, wherein the porous body has a prismatic shape from a first axial location to a second axial location.

14. The distal end of claim 12, wherein the porous body has a plurality of ridges and a plurality of troughs.

15. The distal end of claim 1, wherein the distal end is configured such that, when the distal end is positioned in a body of a patient and the tube is connected to the source of suction, bodily fluids flow through pores of the porous body, through the cavity, and into the tube.

16. A distal end of a medical device comprising:
a tube configured to be coupled to a source of suction;
a porous body coupled to the tube, wherein the distal end includes a cavity between the porous body and the tube; and
a cap positioned at a distal portion of the porous body, such that a proximal surface of the cap is a distalmost end of the cavity.

17. The distal end of claim 16, wherein the cap comprises porous material.

18. The distal end of claim 16, wherein the cavity is configured to receive material from an environment external to the porous body through pores of the porous body.

19. A distal end of a medical device comprising:
a tube configured to be coupled to a source of suction;
a porous body coupled to the tube, wherein the distal end includes a cavity between the porous body and the tube; and
a rib extending from an outer surface of the tube to an inner surface of the porous body, such that the rib defines a first portion of the cavity that is proximal of the rib and a second portion of the cavity that is distal to the rib.

20. The distal end of claim 19, further comprising at least a second rib extending from an outer surface of the tube to the inner surface of the porous body.
